(19) United States
(12) Patent Application Publication
(10) Pub. No.: US 2024/0206944 A1
(43) Pub. Date: Jun. 27, 2024

(54) OPEN LUMEN RADIOFREQUENCY NEEDLE

(71) Applicant: Boston Scientific Medical Device Limited, Galway (IE)

(72) Inventor: Isha Warikoo, Oakville (CA)

(21) Appl. No.: 18/390,499

(22) Filed: Dec. 20, 2023

Related U.S. Application Data
(60) Provisional application No. 63/584,358, filed on Sep. 21, 2023.
(60) Provisional application No. 63/476,878, filed on Dec. 22, 2022.

(51) Int. Cl. A61B 18/12

(52) U.S. Cl. CPC A61B 18/1206; A61B 2018/00601

(57) ABSTRACT

An electrosurgical device for puncturing a tissue includes an elongate body having a proximal portion and a distal portion, and a lumen extending from the proximal portion to the distal portion. The device includes a connector for connecting with an energy source. The distal portion includes an arched distal end, wherein the arched distal end includes a first apex and a second apex. An RF ribbon is configured to deliver energy for puncturing the tissue. The RF ribbon is in electrical communication with the first apex and the second apex.

### TECHNICAL FIELD

The disclosure relates to methods and devices usable to deliver energy within the body of a patient. More specifically, the present invention is concerned with an electrosurgical perforation apparatus.

### BACKGROUND

Devices used for puncturing tissue, for example transseptal tissue of a patient's heart, are typically either mechanical or electrosurgical in nature. Some electrosurgical devices incorporate side-ports and do not have a forward-facing lumen aperture, and consequently lack the ability, for example, to effectively inject fluid or monitor fluid pressure when confined inside of a close-fitting dilator lumen. In addition, while it is possible in some cases for a guide-wire to be passed through or to be received by a side-port, in general, devices lacking a forward-facing aperture do not facilitate the use of a guide-wire with the device. In contrast, devices with a forward-facing aperture are typically more effective in injecting fluid, monitoring pressure, and typically better facilitate usage of a guide-wire than a side-port device.

### SUMMARY

Example 1 is an electrosurgical device for puncturing a tissue. The device includes an elongate body having a proximal portion and a distal portion, and a lumen extending from the proximal portion to the distal portion. The device includes a connector for connecting with an energy source. The distal portion includes an arched distal end, wherein the arched distal end includes a first apex and a second apex. An RF ribbon is configured to deliver energy for puncturing the tissue. The RF ribbon is in electrical communication with the first apex and the second apex.

Example 2 is the device of Example 1, wherein RF ribbon is radiopaque.

Example 3 is the device of Example 1, wherein arched distal end includes a third apex and a fourth apex.

Example 4 is the device of Example 1, wherein the distal portion includes an aperture, and the RF ribbon separates the aperture into equal portions.

Example 5 is the device of Example 1, wherein the distal portion includes an aperture, and the RF ribbon separates the aperture into unequal portions.

Example 6 is the device of Example 1, wherein the elongate body is formed of a first hypotube and a second hypotube.

Example 7 is the device of Example 6, further comprising a tapered socket positioned at a joint reinforcing the first hypotube and the second hypotube.

Example 8 is the device of Example 1, wherein the elongate body is formed of a hypotube that decreases in diameter towards the arched distal end.

Example 9 is the device of Example 1, wherein the RF ribbon is joined to a distal face of the first apex and the second apex.

Example 10 is the device of Example 1, wherein the RF ribbon is joined to a side of the arched distal end.

Example 11 is the device of Example 1, wherein the RF ribbon includes an arc.

Example 12 is the device of Example 1, wherein the arched distal end includes insulation.

Example 13 is the device of Example 12, wherein the insulation is a coating or heat shrink material, and wherein the coating or heat shrink material includes PTFE, Paralene, oxides, nitrides, or ceramics.

Example 14 is the device of Example 1, wherein the arched distal end includes a distal insulated portion and an exposed proximal portion, and the RF ribbon is joined to the exposed proximal portion.

Example 15 is the device of Example 1, wherein the lumen includes an inner surface, and insulation is positioned along the inner surface.

Example 16 is an electrosurgical device for puncturing a tissue comprising: an elongate member defining a lumen for fluid; a distal portion which includes an electrode and a distal face, the electrode being located centrally on the distal face; the distal face defining at least one aperture and including two symmetrical openings located adjacent to the electrode; the distal portion including at least one non-cutting portion and at least one cutting portion configured to deliver energy for puncturing the tissue wherein a distal surface of the electrode forms the at least one cutting portion; and an area of the distal portion defining an arched-taper profile located about the at least one aperture.

Example 17 the device of Example 16, wherein the radiofrequency active plate is permanently affixed to electrically conductive tube the electrically conductive portion of the elongate member.

Example 18 is the device of Example 17, wherein the two symmetrical openings are dimensioned to allow passage of a guidewire.

Example 19 is an electrosurgical device for puncturing a tissue comprising: an elongate member defining a lumen for fluid; a distal portion which includes an electrode and a distal face, the electrode formed by a radiofrequency active plate and permanently affixed centrally upon the distal face; the distal face defining at least one aperture and including two symmetrical openings located adjacent to the electrode, the openings capable of allowing unimpeded passage therethrough; the distal portion including at least one non-cutting portion and at least one cutting portion formed by the radiofrequency active plate for puncturing the tissue; and an area of the distal portion defining an arched-taper profile located about the at least one aperture.

Example 20 is the device of Example 19, wherein the distal face forms an arched-taper profile, and the radiofrequency active plate is located at a distal-most location of the elongate member.

While multiple embodiments are disclosed, still other embodiments of the present invention will become apparent to those skilled in the art from the following detailed description, which shows and describes illustrative embodiments of the invention. Accordingly, the drawings and detailed description are to be regarded as illustrative in nature and not restrictive.

### BRIEF DESCRIPTION OF THE DRAWINGS

In order that the invention may be readily understood, embodiments of the invention are illustrated by way of examples in the accompanying drawings, in which:

FIG. 1 is an illustration of an embodiment of a device including a handle and shaft;

FIGS. 2A-C are illustrations of an embodiment of a device with an electrically conductive tubular member and insulation;

FIGS. 3A-D are illustrations of embodiments of a device with electrically non-conductive coatings on its distal face;

FIGS. 4A and 4B illustrate an embodiment with a rectangular-shaped protruding electrode;

FIGS. 5A and 5B illustrate an embodiment with a star-shaped (or pie cutter-shaped) protruding electrode;

FIG. 6 illustrates an embodiment of an electrosurgical device having an exposed surface;

FIGS. 7A and 7B illustrate a method of puncturing tissue within a heart using the electrosurgical device of FIG. 6;

FIG. 8 shows a device including a handle and shaft in accordance with an embodiment of the present invention;

FIG. 9 illustrates the embodiment of FIG. 8 in the context of its use for puncturing tissue within a heart;

FIG. 10 is a close-up perspective view of the distal end of the embodiment of FIG. 8 without its insulative layer;

FIG. 11 is an end view of the embodiment of FIG. 8;

FIG. 12 is a cross-sectional view of the embodiment of FIG. 8 as shown in FIG. 11 taken along line 5-5;

FIG. 13 is a close-up perspective view of the distal end of the embodiment of FIG. 8 with its insulative layer partially removed;

FIG. 14 is a close-up perspective view of the distal end of another embodiment in accordance with the present invention and with its insulative layer partially removed;

FIGS. 15A and 15B illustrate an embodiment of an electrosurgical device with an arched distal end;

FIGS. 16A and 16B illustrate an additional embodiment of an electrosurgical device with an arched distal end;

FIGS. 17A and 17B illustrate a additional embodiment of an electrosurgical device with an arched distal end;

FIG. 18 illustrates an embodiment of an electrosurgical device with multiple arched RF ribbons;

FIGS. 19A and 19B illustrate an embodiment of a method of puncturing tissue within a heart;

FIG. 20 depicts a side view of an embodiment of a technical feature (an option) that may be included with any of the elongated medical needle assemblies disclosed herein; and

FIG. 21 illustrates the technical feature of FIG. 20 that can strengthen the joint between the two separate hypotubes in accordance with an embodiment of the disclosure.

While the invention is amenable to various modifications and alternative forms, specific embodiments have been shown by way of example in the drawings and are described in detail below. The intention, however, is not to limit the invention to the particular embodiments described. On the contrary, the invention is intended to cover all modifications, equivalents, and alternatives falling within the scope of the invention as defined by the appended claims.

### DETAILED DESCRIPTION

A conventional Brockenbrough transseptal needle with a sharp beveled tip has a forward-facing aperture that may be used for injecting fluid or monitoring pressure. However, conventional transseptal needles typically utilize mechanical force to puncture tissue, which is less effective at puncturing tissue under certain circumstances and poses potential increased risks to the patient as a result of skiving, and/or puncturing unintended anatomy. To meet the challenge of puncturing through a tissue that does not facilitate being mechanically punctured, some physicians have used an electrical generator or the like to electrify the mechanical needle and to thereby produce an ad hoc electrosurgical device with a forward-facing aperture. One drawback to electrifying a Brockenbrough needle is the risk of tissue coring. A core (or plug) of tissue is typically cut from surrounding tissue upon delivery of energy and is subsequently captured in the lumen of the electrosurgical device upon advancement of the needle through tissue. The tissue core may be released from the lumen by flushing, potentially leading to emboli and increasing the risk of a stroke or some other ischemic event. Furthermore, a non-insulated and electrified Brockenbrough needle bears an additional increased risk of burns to the patient and physician.

This disclosure includes different embodiments of an electrosurgical device that has a distal face for creating an elongate initial puncture that is configured to be dilated when the device is advanced while reducing the risks of tissue coring and emboli formation. The elongate initial puncture in the context of the present invention shall be understood to be a slit-like incision that forms an opening in human tissue. Embodiments of the device also have a forward-facing lumen aperture to provide for pressure monitoring, forward fluid delivery, and to facilitate being used with a guidewire.

In typical embodiments, the distal surface of an electrode defines at least one elongate portion (when seen from the end view), whereby the device creates a puncture corresponding with at least one elongate portion thereby defining one or more flaps of tissue which the distal face of the device may push aside when the device is advanced. The term elongate electrode is used to describe electrodes that are non-circular and that may be described as being longer in one dimension than in another. In some embodiments, the distal surface of the electrode defines an elongate shape which is generally C-shaped, U-shaped, semicircular-shaped, shaped like a segment of a circle, shaped like an arc of a circle, arcuate, crescent-shaped, rectangular-shaped, generally straight, or star-shaped (i.e. having segments radiating from a central point). Some embodiments have a pair of generally parallel electrodes which are generally straight (or rectangular-shaped) and operable for bi-polar delivery of energy. With particular regard to some embodiments, the distal surface of the electrode defines an elongate shape which is generally a thin, rectangular band or plate. Furthermore, some embodiments are configured such that the thin radiofrequency active electrode plate straddles a lumen aperture at a central location on the distal end of the device. The electrode does not completely encircle or enclose the forward-facing lumen aperture, thereby avoiding having a ring-shaped electrode that may possibly core tissue. Moreover, the shape and placement of the electrode does not substantially obstruct the forward-facing lumen aperture and therefore enables passage of any element(s) such as, but not limited to, fluid or a guidewire through the forward-facing lumen aperture.

While this disclosure describes electrosurgical devices that are generally circular in cross-section, the concepts and claims of this disclosure also apply to non-circular devices e.g. square-shaped, elliptical-shaped. Furthermore, some embodiments are configured such that an electrode used for puncturing tissue does not completely encircle or enclose a forward-facing lumen aperture, thereby avoiding having a ring-shaped electrode that may possibly core tissue.

Thus, the present inventors have conceived and reduced to practice a surgical device for puncturing tissue, such as an atrial septum of a heart, wherein the surgical device allows for forward fluid delivery for staining the septum and has less risk of coring tissue relative to an electrified Brockenbrough needle or similar device. The device comprises a distal face defining at least one aperture, with the distal face including at least one cutting portion and non-cutting portions cooperating to produce an elongated cut in a tissue when electrical energy is delivered to the distal face, while avoiding coring of the tissue. Typical embodiments can be advanced over a guide-wire to a treatment site.

With specific reference now to the drawings in detail, it is stressed that the particulars shown are by way of example and for purposes of illustrative discussion of certain embodiments of the present invention only. Before explaining embodiments of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

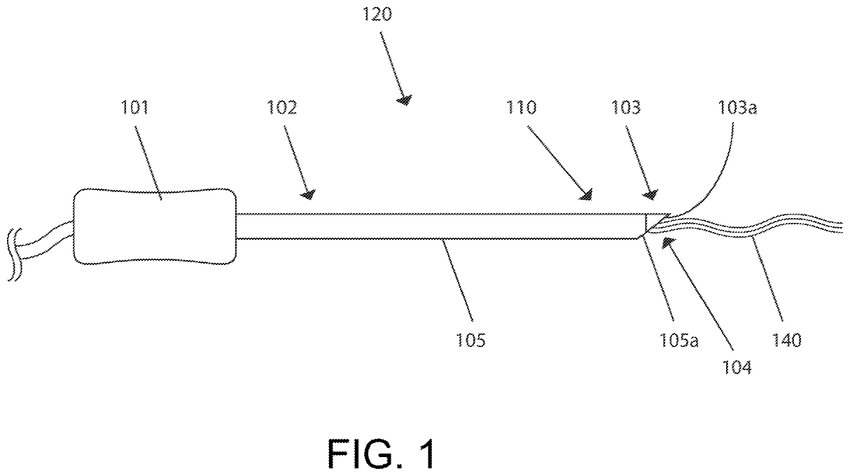

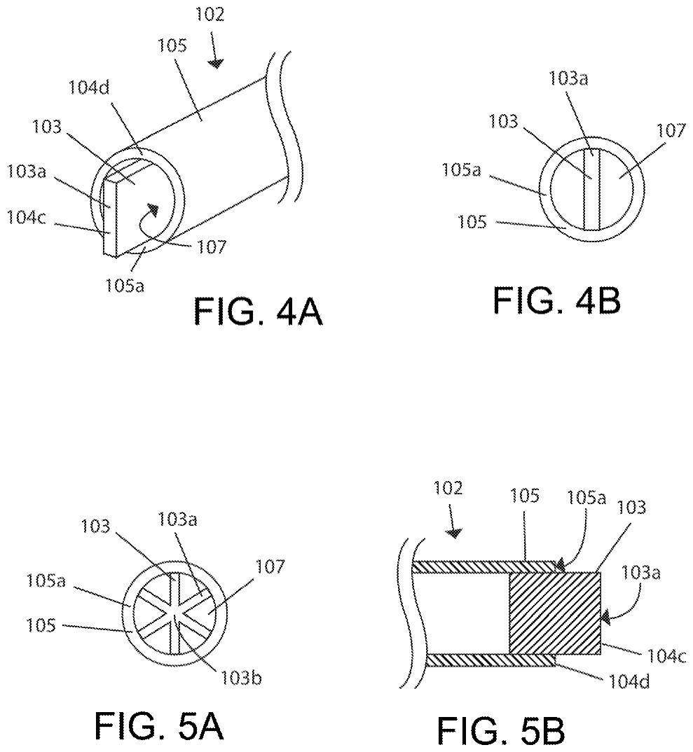

FIG. 1 is an illustration of an embodiment of a device including a handle and shaft. Electrosurgical device 120 of FIG. 1 is comprised of elongate member 102, electrically insulating material 105 and distal portion 110. The handle 101 is mechanically coupled to the proximal end of the elongate member 102. The elongate member 102 may include a taper that reduces from the proximal end toward the distal portion 110. Elongate member 102 defines a lumen (FIG. 4A). Distal portion 110 includes electrode 103 and distal face 104 (further described herein below) which defines an aperture. The embodiment is operable to direct a fluid forward, as represented by fluid flow lines 140. The forward-facing aperture facilitates the device being used with a guide-wire.

Some embodiments of electrosurgical device 120 include electrically insulating material 105 covering portions of the shaft of elongate member 102 and/or distal face 104 of the device. The insulating material is understood by one skilled in the art to be an effective insulator, which may be a 100 percent insulating material or a partially insulating material. In the case of a layer of partially insulating material being located on distal face 104, the partially insulating material functions as an effective insulator, when the device is used, by only allowing limited electrical energy flow through the partially insulating material, such that there is insufficient electrical energy to heat adjacent tissue to create a void in the tissue for advancing the electrosurgical device through.

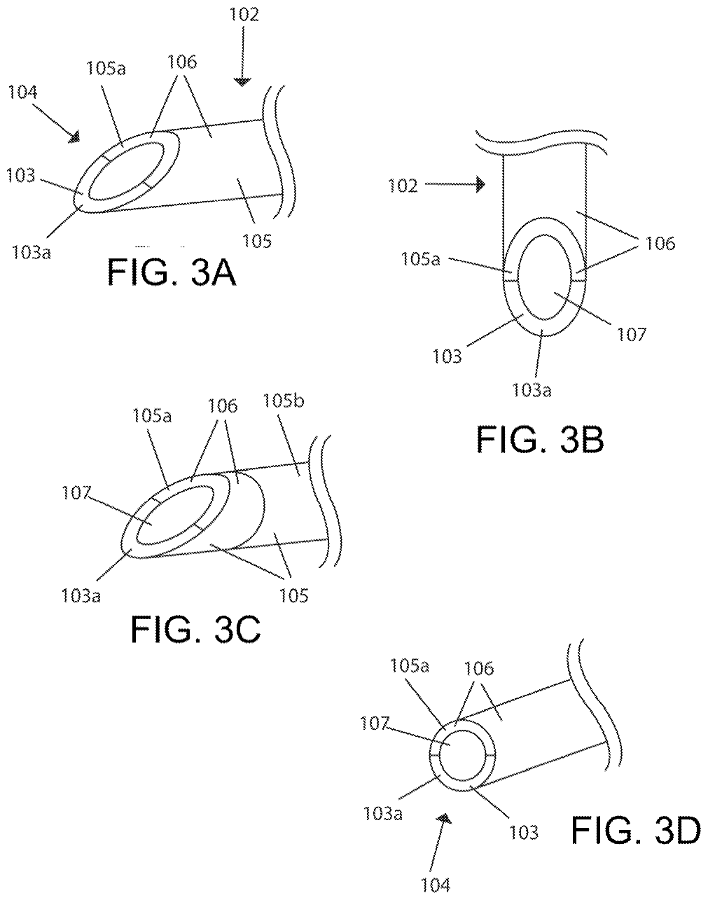

In general, in this disclosure, the term “distal face” is with reference to the entire electrosurgical device and used to refer to the end surfaces of the device seen from the distal end view (not interior or side surfaces). The term “distal surface” is used to refer to the end surfaces seen from the distal end for a particular part of the device. In some embodiments, the distal surface of elongate member 102 and the distal face 104 refer to the same surface, for example, the embodiment of FIG. 3A.

Various embodiments of this disclosure include an electrosurgical device 120 for puncturing tissue comprising: an elongate member 102 defining a lumen 111 for receiving a fluid; with distal face 104 of the electrosurgical device defining at least one aperture 107; and the distal face 104 including at least one cutting portion 103a and at least one non-cutting portion 105a cooperating to produce an elongated cut in a tissue when electrical energy is delivered to the distal face 104 while avoiding coring of the tissue. Some embodiments only have one distal aperture, while other embodiments have more than one aperture. In some examples, the device can be described as having an aperture that is divided into more than one portion.

Various embodiments of this disclosure further include at least one cutting portion 103a being configured to create an initial partial puncture upon energy delivery, the initial partial puncture substantially corresponding to the at least one cutting portion. The “initial partial puncture” is a puncture created by energy delivery before the tissue is dilated or pushed aside when the electrosurgical device is advanced after energy delivery;

the initial partial puncture is too small to receive the device without dilating or pushing aside tissue. As previously noted, distal face 104 is configured for advancing while avoiding coring tissue during advancement of elongate member 102. The initial puncture is dilated by distal face 104 of electrosurgical device 120 as the device is advanced; if the shaft of the elongate member is tapered there is typically further dilation by the shaft during advancement.

In some embodiments, elongate member 102 has a length of about 30 cm to about 100 cm to facilitate the puncture of a septum of a heart. In some embodiments, the elongate member has an outer diameter of about 0.40 mm to about 1.5 mm to minimize hemodynamic stability, for example, by ensuring that the perforation will not cause hemodynamic instability once electrosurgical device 120 is removed. In some embodiments, the electrosurgical device 120 is a stiff elongate needle.

Some embodiments of electrosurgical device 120 include an elongate member 102 having flexural rigidity of at least about 0.016 Nm2, for example a flexural rigidity of about 0.017 Nm2, to provide tactile feedback for a user of the device.

Some embodiments of the device have markers for highlighting the location of important landmarks on electrosurgical device 120. Such landmarks may include the location where the elongated member 102 begins to curve, the location of the electrode 103, or the location of the proximal edge of a beveled distal face. In some embodiments the marker is radiopaque. Imaging markers may be different shapes including, but not limited to, a ring-shaped hollow band or a coil. Alternative embodiments include imaging markers that are disc-shaped, rectangular, and elongate, that define other geometric shapes, or that define symbols.

An elongate member 102, which can be comprised of one or more layers/components of plastic, other polymers, metal, or other materials, may have a marker embedded in its sidewall which may be either all metal or substantially (mostly) metal. For example, the marker receiving sidewall can be covered with a relatively thin layer of polymer, such as the sidewall being covered with a layer of electrical insulation. As all metals are radiopaque to some degree, a radiopaque marker should be more radiopaque than the metal comprising the elongate member to function properly. In general, for any embodiment of the device having a radiopaque marker, the radiopaque marker may be comprised of a material that is more radiopaque than whatever material elongate member 102 is comprised of.

While the embodiment of FIG. 1 has a generally straight elongate member 102, in alternative embodiments, the elongate member comprises a curved section. In some examples, the curved section has a curve length of about 10 to about 25 cm and traverses from about 20° to about 40° of a circle. In some other examples, the curved section has a curve length of from about 4 to about 7 cm and traverses from about 70 degrees to about 110 degrees of a circle.

Typically, handle 101 comprises a connector for receiving an electrical plug or other electrical connector, and a fluid port for receiving a second connector, for example, a luer lock. Electrical energy may be delivered from an energy source, through the connector and, typically, a wire (not shown in the drawing) located within handle 101. The electrical energy is then conveyed to the elongate member 102 and electrode 103.

Some embodiments of the handle 101 include a relatively large graspable surface having ridges so that tactile feedback can be transmitted relatively efficiently, for example by transmitting vibrations.

In some embodiments, the handle 101 allows users to navigate through patient anatomy and guide a distal tip of the electrosurgical device 120 to a puncture site with greater ease. In some aspects, the handle 101 may include a curve indicator. The curve indicator points to the direction in which the needle is curving, which allows the user to maneuver the device accordingly.

In some embodiments, one end of a tubing is operatively coupled to a source of fluid (not shown in drawing), for example a syringe, pump, intravenous fluid bag, etc., and the other end of the tubing is operatively coupled with a connector to a fluid port of handle 101 which is in fluid communication with lumen 111 of elongate member 102 via a conduit in the handle (not shown), whereby the tubing and lumen 111 are in fluid communication with one another, thus allowing for a flow of fluid between an external device and lumen 111.

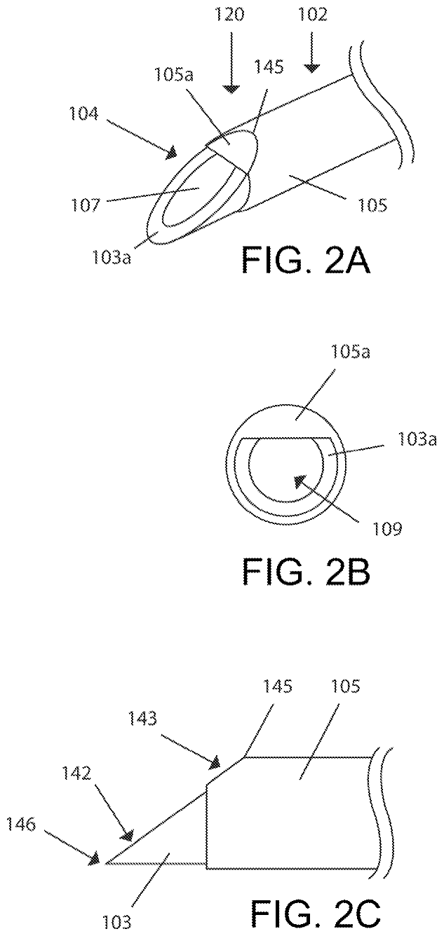

In some embodiments, aperture 107 and the lumen 111 (e.g. FIG. 2A to 2C) together provide a pressure transmitting lumen which is coupled to the external tubing by a connector, and the tubing is in fluid communication with a pressure sensing device, for example, a pressure transducer.

FIGS. 2A to 2C illustrate the distal portion of an embodiment of an electrosurgical device 120 in which elongate member 102 is an electrically conductive tubular member. Elongate member 102 defines a lumen 111 for receiving a fluid. The fluid within the lumen (FIG. 2B) may be injected, withdrawn, or may remain substantially stationary. In some embodiments, the electrically conductive tubular member is comprised of stainless steel.

The electrically conductive tubular member is at least partially covered by electrically insulating material 105 with a distal portion of the electrically conductive tubular member uncovered (i.e. electrically exposed) to define electrode 103. The non-cutting portion 105a of the distal face comprises a layer of electrical insulation, which in some embodiments (e.g. FIGS. 2A to C), is the same as the electrically insulating material 105 covering the shaft of the tubular member, which includes both the electrically insulating material 105 covering the shaft of the tubular member extending over the distal face 104 and the electrical insulation covering the distal face 104 being the same type of material applied separately. In alternative embodiments, the layer of electrical insulation covering distal face 104 is a different type of insulation.

Distal face 104 of the electrosurgical device defines an aperture 107 which is in communication with lumen 111. Referring to FIG. 2B, the layer of electrical insulation (non-cutting portion 105a) has the shape of a segment of a circle whereby the electrically conductive tubular member (cutting portion 103a of FIG. 2B) and the layer of electrical insulation define aperture 107.

In the embodiment of FIGS. 2A to 2C, distal face 104 is beveled and is comprised of an electrically exposed and conductive cutting portion 103a and an electrically insulated non-cutting portion 105a. The distal surface of electrode 103 forms cutting portion 103a which, in this embodiment, is generally C-shaped or arcuate shaped when viewing the distal face 104 from a distal end-view. Cutting portion 103a is elongate i.e. it is non-circular and has a length greater than its width. Furthermore, cutting portion 103a does not completely encircle, circumscribe or enclose aperture 107 but rather partially surrounds the aperture.

The proximal portion 143 of distal face 104 (FIG. 2C) is comprised of non-cutting portion 105a. Electrically insulated portion 105a extends from a periphery 145 of distal face 104 to partially cover the end surface of the tubular member. In some embodiments, non-cutting portion 105a is comprised of polymer insulation, which may be a heat shrink, a spray coating, or a material selectively coated by vapor deposition. In some alternative embodiments, non-cutting portion 105a comprises a ceramic. In some embodiments, the distal face of the electrically conductive tubular member has a step recess wherein a layer of insulation is received to thereby provide for a planar distal face 104 (i.e. to avoid having a stepped surface).

The cutting portion 103a is configured such that, when the electrosurgical device is advanced into a tissue, energy delivered by the electrically exposed cutting portion 103a punctures the tissue without the tissue substantially occluding lumen 111. In particular, it is the leading surface of electrode 103 that defines the cutting surface of the electrode (i.e. cutting portion 103a) which actually cuts into tissue when the energy delivery device is advanced while delivering energy. The outer perimeter of the distal surface of electrode 103 defines a portion (but not all) of the perimeter of distal face 104 (FIG. 2A), whereby the device creates a puncture corresponding with a portion (but not all) of the perimeter of the distal face 104, such that the puncture defines a flap of tissue which the beveled distal face pushes aside as the device is advanced.

The embodiment of electrosurgical device 120 of FIG. 2C includes a distal tip 146 which is substantially rounded or atraumatic, as it is not necessary to have a sharp tip on the device for puncturing. The rounded tip reduces the risk of accidental tissue puncture and skiving of supporting dilators. In other words, the distal portion 142 of the distal face is substantially rounded. In some alternative embodiments, the tip of the device is sharp. Furthermore, the planar surface of distal face 104 is substantially atraumatic.

While in the embodiment of FIGS. 2A to 2C, the distal face is beveled, in some alternative embodiments the distal face comprises a flat tip. In such embodiments, the configuration of the distal face allows electrosurgical device 120 to be operable to electrically puncture and push aside tissue without coring, as the device is advanced.

FIGS. 3A to 3D illustrate embodiments of electrosurgical device 120 wherein an electrically conductive material forms cutting portion 103a and non-cutting portion 105a comprises an electrically insulative coating 106 on the distal face of the device. In each of FIGS. 3A to 3D the distal surface of elongate member 102 includes one cutting portion 103a and one non-cutting portion 105a. Alternative embodiments contain more than one cutting portion 103a and/or more than one non-cutting portion 105a. In some embodiments the electrically insulative coating 106 comprises a non-polymeric layer of a material selected from the group including oxides, nitrides and ceramics. More specific examples include the layer of material being a metal oxide, silicon oxide, silicon dioxide, or diamond thin film. In other embodiments, the electrically insulative coating 106 may be any solid state insulating material.

In some embodiments elongate member 102 comprises an electrically conductive tubular member (e.g. stainless steel), and the at least one non-cutting portion 105a comprises the electrically insulating material positioned along a portion of a distal surface of the elongate member 102, and furthermore an electrically exposed portion of the distal surface of the elongate member 102 forms the at least one cutting portion 103a. Such embodiments may be produced by a layer of electrically insulative oxide being deposited upon an electrically conductive metal tube by methods including (but not limited to) evaporation, chemical vapor deposition, or sputtering. This layer can be deposited on only the distal surface of the tube or it can also be deposited on the side of the tube. A portion, or portions, of the electrically insulative coating 106 is removed by methods including (but not limited to) laser ablation, chemical etching or plasma etching to form the at least one cutting portion 103a. Alternatively, masking can be used to cover the at least one cutting portion 103a during the deposition process and the masking removed after deposition to expose the electrode, while the rest of the distal surface is covered with insulative material to form at least one non-cutting portion 105a.

FIGS. 3A and 3B are side and front perspective views, respectively, of an electrosurgical device 120 wherein the distal face 104 comprises a beveled surface. Non-cutting portion 105a and electrical insulation 105 (on the shaft of elongate member 102) are both comprised of the electrically insulative coating 106. Cutting portion 103a is comprised of the distal surface of electrode 103. In the embodiment of FIG. 3C, the distal portion of the electrically insulating material 105 on the shaft of elongate member 102 is comprised of the electrically insulative coating 106 (described above) and the proximal portion is comprised of polymer 105b. In the embodiment of FIG. 3D, the distal face 104 of the device comprises a substantially flat tip.

In some alternative embodiments, the at least one cutting portion is located on the distal face 104 along an inner surface of the elongate member 102 i.e. the cutting portion 103a is adjacent aperture 107 while not extending to the outer periphery of the distal face 104.

Non-polymeric coatings disclosed above (e.g. ceramics, oxides, and diamond thin film) can function as effective insulators in thinner layers than typical polymers. In some examples of electrosurgical device 120, the electrically insulative coating comprises a layer less than about 1 micron thick. In some specific examples, the electrically insulative coating comprises a layer from about 100 nanometers to about 1 micron thick. In some other examples, the electrically insulative coating comprises a layer about 1 micron to about 50 microns thick. In some specific examples, the electrically insulative coating comprises a layer about 1 micron to about 25 microns thick, and some more specific examples, the electrically insulative coating comprises a layer about 1 micron to about 10 microns thick.

In some alternative embodiments, wherein the at least one cutting portion comprises an electrically conductive material, the at least one non-cutting portion of the distal face is comprised of a partially electrically insulating layer. A flow of electricity through an electrode that causes enough tissue heating to puncture tissue electrically (i.e. without a pushing force), when applied to an effective partially insulating layer on distal face 104 of the device, results in some electrical flow through the partially insulating layer, but it is insufficient to heat the tissue to create a void in the tissue for advancing the electrosurgical device through

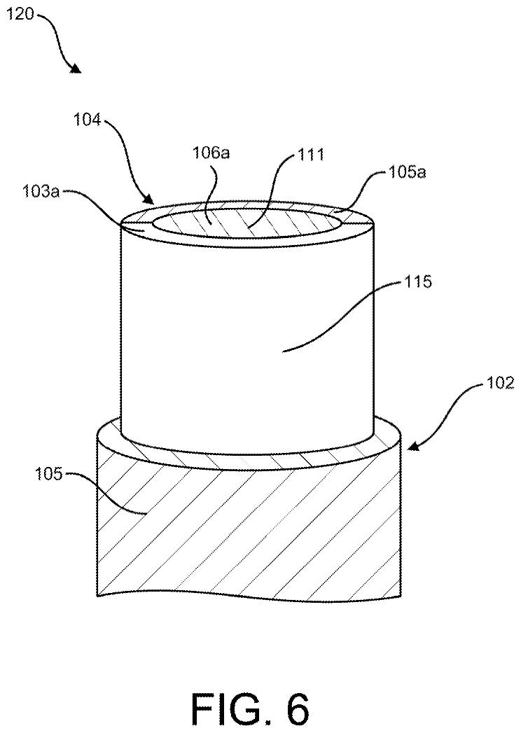

The related embodiments of FIGS. 4A, 4B, 5A, and 5B are for an electrosurgical device 120 comprising a protruding electrode 103 defining a leading surface 104c (FIGS. 4A and 5B) distal of the elongate member 102, with the leading surface 104c including the at least one cutting portion 103a. Distal face 104 comprises a trailing surface 104d (FIGS. 4A and 5B) defined by a distal end surface of the elongate member 102. Trailing surface 104d comprises an electrically insulating material 105 to form non-cutting portion 105a. In some embodiments, leading surface 104c is substantially flat. In some examples, protruding electrode 103 is connected to a rotary mechanism such that the leading surface 104c may be rotated when energy is delivered. Distal face 104 of the electrosurgical device includes leading surface 104c and trailing surface 104d.

In the embodiment of FIGS. 4A and 4B, protruding electrode 103 substantially bisects the aperture 107 into two parts. The protruding electrode 103, when seen in end view, is substantially rectangular-shaped. In some examples, the leading surface 104c is substantially rectangular-shaped.

Some embodiments of electrosurgical device 120 include the protruding electrode 103 comprising at least three elongate portions radiating from a center point 103b. Some such devices include the protruding electrode 103 substantially dividing the aperture 107 into at least three pie slice shaped wedges. Some embodiments include protruding electrode 103 defining leading surface 104c as having at least three elongate portions radiating from a center point 103b. Some embodiments include six elongate portions of electrodes 103 radiating from a center point 103b to divide aperture 107 into to six wedge-shaped segments. Some embodiments further include the at least three elongate portions of the leading surface 104c sloping proximally as they radiate from the center point 103b.

Some embodiments of FIGS. 4A, 4B, 5A, and 5B include an elongate member 102 comprising an electrically conductive tubular member with an electrically insulating material 105 on the tubular member's distal surface to form non-cutting portion 105a. Some alternative embodiments include elongate member 102 comprising a non-conductive material, for example, polymer.

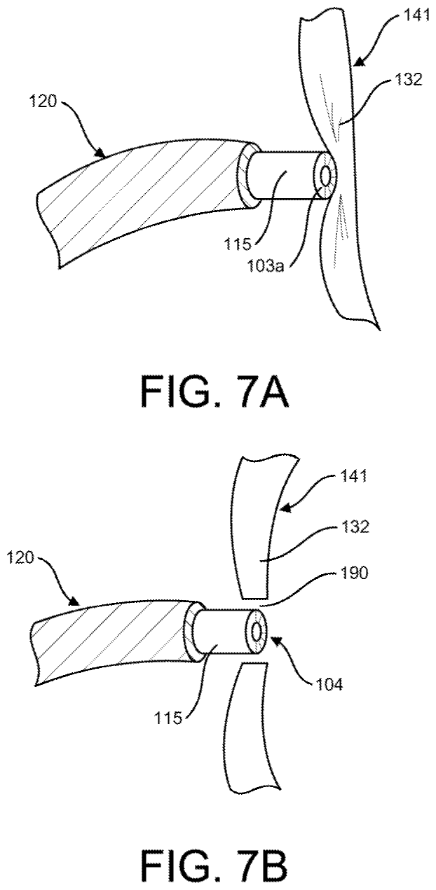

FIG. 6 illustrates an embodiment of an electrosurgical device 120 similar to the electrosurgical device 120 of FIG. 3D. The distal face 104 of the electrosurgical device 120 is atraumatic and forms a circle. A first half of the circle forms the cutting portion 103a of the electrode, while the other half of the face is insulated and forms the non-cutting portion 105a. The non-cutting portion is coated in an insulative material to prevent tissue coring and mitigate the risk of causing an embolism. An inner surface insulative coating 106a may cover the inside of the lumen 111 to prevent an electronic signal from being transmitted along the entire distal face 104.

The elongate member 102 can be covered in insulation 105 along a majority thereof, with an exposed distal portion 115 extending to the distal face 104. The insulation 105 may include a polymeric or non-polymeric coating as discussed above. For example, the insulation 105 may include a PTFE heat-shrink or Paralene dielectric coating. In FIG. 6, insulated portions of the electrosurgical device 120 are illustrated by cross-hatching while uninsulated active regions do not include cross-hatching.

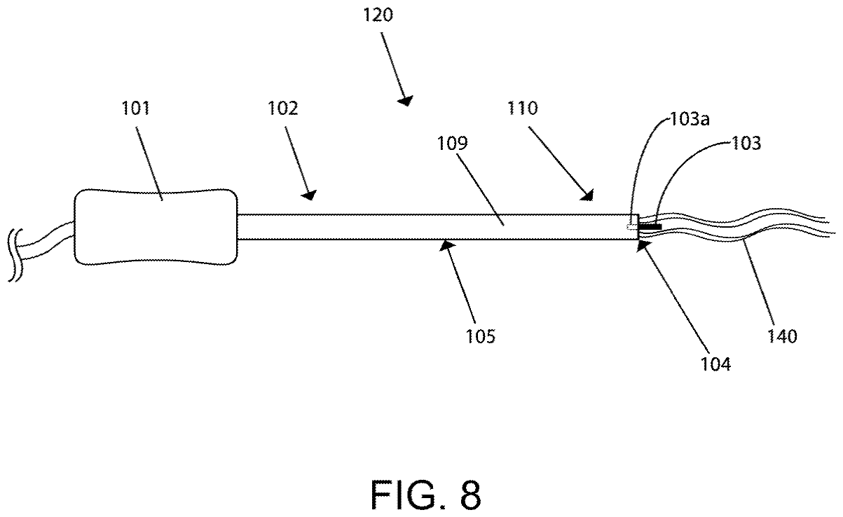

FIGS. 7A and 7B illustrate an embodiment of a method of puncturing tissue using the electrosurgical device 120 of FIG. 6. The method comprises the steps of tenting the tissue 141, for example an arial septum 132, with the distal face 104 of the electrosurgical device as shown in FIG. 7A. During tenting, energy is delivered through the electrically exposed conductive cutting portion 103a of electrosurgical device 120 to the tissue 141. This creates a puncture 190 substantially corresponding to the cutting portion 103a of the distal face 104 of the electrosurgical device 120. Once a puncture 190 is created, the puncture 190 can be dilated or widened by advancing the electrosurgical device through the puncture 190 without coring the tissue 141. In some embodiments, a dilator may be use to dilate or widen the puncture 190. In FIGS. 7A and 7B, insulated portions of the electrosurgical device 120 are illustrated by cross-hatching while uninsulated active regions do not include cross-hatching.

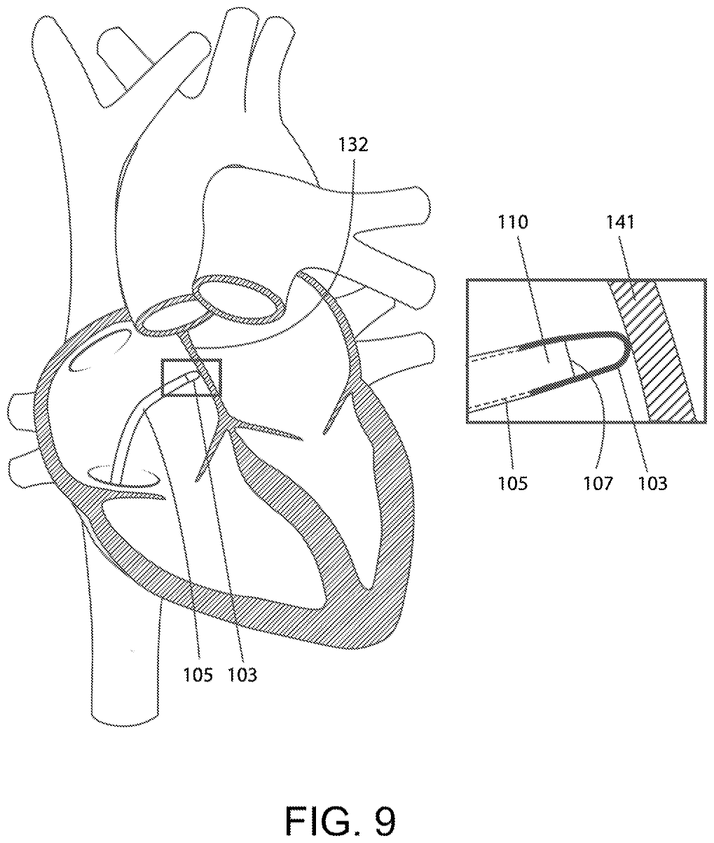

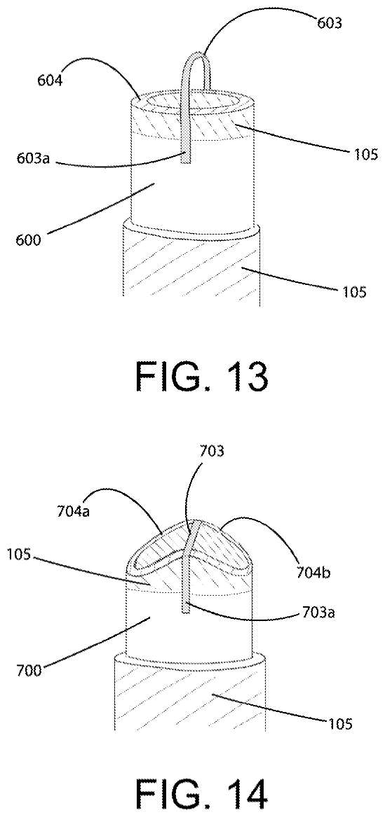

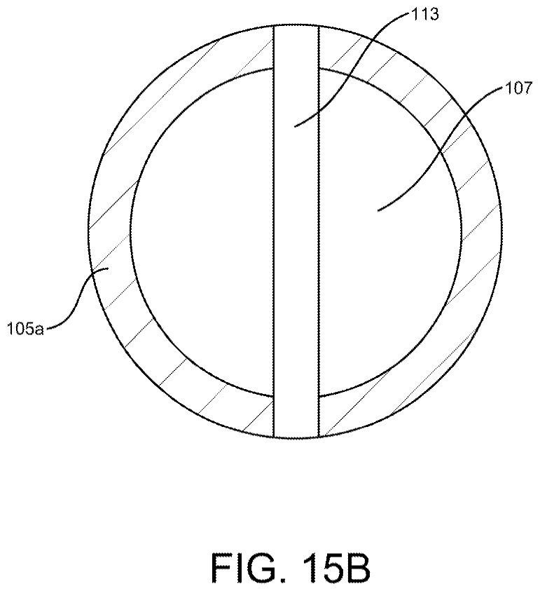

FIG. 8 is an illustration of an embodiment of an electrosurgical device 120 including a handle 101 and shaft 109. The electrosurgical device 120 of FIG. 8 includes an elongate member 102, electrically insulating material 105 and distal portion 110. The handle 101 is mechanically coupled to the proximal end of the elongate member 102. The elongate member 102 defines a lumen (visible as element 111 in FIG. 12). The distal portion 110 includes an electrode 103 and distal face 104 (further described herein below) which defines an aperture (visible as element 107 in FIG. 10). The electrode 103 is attached to the shaft 109 at attachment portions 103a shown in dotted line as the attachment occurs under the electrically insulating material 105. It should be understood that the lumen provides a passageway completely through the elongate member 102 from the handle 101 to the aperture surrounded by the distal face 104. The embodiment is operable to direct a fluid forward as shown and represented by fluid flow lines 140. The forward-facing aperture also facilitates the device being used with a guidewire. It should be readily understood that either fluid or guidewire would therefore pass through the lumen and aperture unimpeded by the electrode.

Some embodiments of electrosurgical device 120 include electrically insulating material 105 covering portions of the shaft of elongate member 102 and/or distal face 104 of the device. The insulating material is understood by one skilled in the art to be an effective insulator, which may be a 100 percent insulating material or a partially insulating material. In the case of a layer of partially insulating material being located on distal face 104, the partially insulating material functions as an effective insulator, when the device is used, by only allowing limited electrical energy flow through the partially insulating material, such that there is insufficient electrical energy to heat adjacent tissue to create a void in the tissue for advancing the electrosurgical device through. The insulating material may, for example, be formed from paralene dielectric coating or similar material to prevent transmission of radiofrequency energy. The full length of the shaft may be covered in a suitable material such as polytetrafluoroethylene (PTFE) heat-shrink, which acts as insulation. The PTFE insulation covers the entire length of the needle, excluding the distal end of the shaft and electrode. This material is highly lubricious, allowing easy advancement/retraction of the device from accessory devices and patient vasculature. It should be readily understood that different types of insulation material may be used together in conjunction with one another and may be selected based upon material properties such as, but not limited to, dielectric characteristics, durability, and flexibility.

Various embodiments of this disclosure include an electrosurgical device 120 for puncturing tissue comprising: an elongate member 102 defining a lumen 111 for receiving a fluid; with a distal portion 110 of the electrosurgical device defining at least one aperture 107; and the distal portion 110 including at least one cutting portion (e.g., electrode 103) and a distal face 104 cooperating to produce an elongated cut in a tissue when electrical energy is delivered to the cutting portion 103 while avoiding coring of the tissue. Some embodiments may only have one distal aperture, while other embodiments may have more than one aperture. In some examples, the device may be described as having an aperture that is divided into more than one portion. Indeed, the cutting portion forming the electrode 103 is centrally located at the aperture in such a manner that the distal-most tip of the device 120 is sectioned into two distinct and symmetrical openings where each of the openings are adjacent to the electrode 103.

Various embodiments of this disclosure further include a cutting portion 103 being configured to create an initial partial puncture upon energy delivery, the initial partial puncture substantially corresponding in shape to the distal facing surface of the cutting portion. The “initial partial puncture” is a puncture created by energy delivery before the tissue is dilated or pushed aside when the electrosurgical device is advanced after energy delivery. It should be understood that the initial partial puncture is too small to receive the device without dilating or pushing aside tissue. As previously noted, distal face 104 is configured for advancing while avoiding coring tissue during advancement of the elongate member 102. The initial puncture is dilated by the distal face 104 of the electrosurgical device 120 as the device is advanced. Though not shown, it should be readily understood that if the shaft 109 of the distal portion 110 is tapered there is typically further dilation by the shaft 109 during advancement.

In some embodiments, the elongate member 102 may have a length of about 30 cm to about 100 cm to facilitate vascular entry from a patient's neck or groin in order to access and subsequently puncture of a septum of a heart. In some embodiments, the elongate member has an outer diameter of about 0.40 mm to about 1.5 mm to minimize hemodynamic stability, for example, by ensuring that the perforation will not cause hemodynamic instability once the electrosurgical device 120 is removed. In some embodiments, the electrosurgical device 120 is a stiff elongate needle.

An elongate member 102, which may be comprised of metal, or other electrically conductive materials, may have a marker embedded in its sidewall which may be either all metal or substantially (mostly) metal. For example, the marker receiving sidewall may be covered with a relatively thin layer of polymer, such as the sidewall being covered with a layer of electrical insulation. As all metals are radiopaque to some degree, a radiopaque marker should be more radiopaque than the metal comprising the elongate member to function properly. In general, for any embodiment of the device having a radiopaque marker, the radiopaque marker may be comprised of a material that is more radiopaque than whatever material forms the elongate member 102. Accordingly, it should be readily apparent that the elongate member is effectively an electrically conductive tube.

While the embodiment of FIG. 8 as illustrated includes a generally straight elongate member 102, in alternative embodiments, the elongate member may be a curved section. In some examples, the curved section has a curve length of from about 10 cm to about 25 cm and traverses from about 20° to about 40° of a circle. In some other examples, the curved section has a curve length of from about 4 cm to about 7 cm and traverses from about 70 degrees to about 110 degrees of a circle.

Typically, the handle 101 includes a connector for receiving an electrical plug or other electrical connector, and a fluid port for receiving a second connector, for example, a Luer-type lock. Electrical energy may be delivered from an energy source as is known in the art and not further described herein. Such energy source providing radiofrequency energy, through the connector and, typically, a wire (not shown in the drawing) located within the handle 101. The radiofrequency energy is then conveyed to the elongate member 102 and electrode 103.

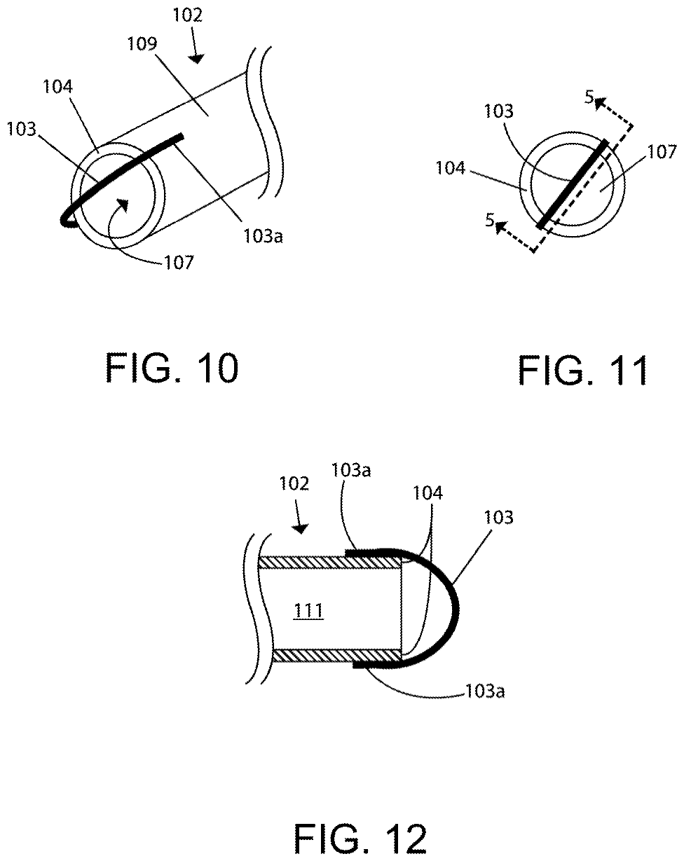

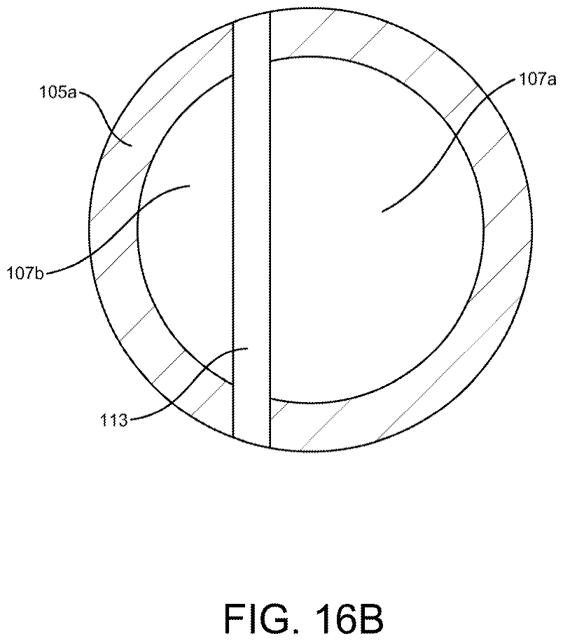

FIG. 9 illustrates the present invention in the context of its intended use within a patient. More specifically, radiofrequency energy is delivered through the electrically exposed conductive portion formed by the electrode 103 of the electrosurgical device 120 to tissue 141 at a target site for creating a puncture substantially corresponding to an elongate cutting portion of the electrode 103 of the electrosurgical device 120. It should be understood that dilating or widening the puncture primarily by advancing the distal face 104 of the electrosurgical device 120 may occur without coring the tissue. In some embodiments such as shown and later described with regard to FIG. 14, delivering energy includes creating a slit in the tissue and dilating or widening is completed without further delivery of energy. In some embodiments, the target site is a tissue within a heart, and in some particular embodiments the tissue is an atrial septum 132. Though shown without a sheath, it should be understood that the present invention may be used in conjunction with a sheath, for example, a dilator sheath. The term dilate is used herein to mean “to make wider, larger, or more open”. Dilating the puncture typically includes displacing the tissue. In some embodiments dilation includes wedging apart and thereby outwardly compressing surrounding portions of the tissue.

In some embodiments, the distal surface of the electrically exposed conductive portion is generally flat plate-like ribbon or band. Advantageously, the arched profile (as seen in the close-up portion of FIG. 9) of the distal end of the electrode 103 acts as a taper, gradually expanding the slit-shaped opening when crossing the septum. This removes the risk of coring, which otherwise would introduce free-floating particles of tissue that would cause an embolism. As the distal tip of the shaft is blunt, this ensures there is no risk of trauma to patient anatomy and no risk of dilator skiving. Further, the active electrode plate is radiopaque, which allows for visualization under fluoroscopy/echocardiography. This also allows physicians to target the desired site on the septum.

With reference to FIG. 10, the embodiment of FIG. 8 is shown in a close-up perspective view of the distal end of the elongate member 102 without its insulative layer. The insulative layer is removed to reveal lateral attachment portions 103a where the band forming the electrode 103 attaches to the electrically conductive shaft 109. The shaft 109 may be composed of 304 stainless steel as is commonly used to manufacture transseptal access puncture devices. Additionally, this material is biocompatible, conductive and possess suitable material properties (e.g., stiffness) for implementation of the present invention. The electrode 103 including the lateral attachment portions 103a may be composed entirely of platinum or composed of 304 stainless steel plated with platinum. The use of platinum, or an equivalently suitable material, ensures that the electrode is radiopaque and may be visualized under fluoroscopy and echocardiography. The lateral attachment portions 103a may be fixed to the distal shaft using micro laser welding or similar techniques thereby permanently securing the electrode 103 to the electrically conductive shaft 109. The plate may include a width that is a minimum of 0.008 inches, which may concur with the distal shaft wall thickness (seen in cross section of FIG. 12).

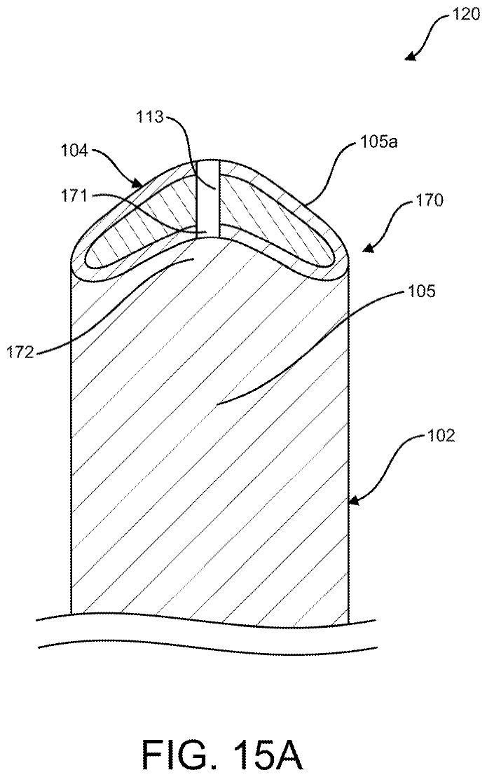

With reference to FIG. 11, there is shown an end view of the device 120 as shown in FIG. 10. Here, the space to either side of the electrode 103 clearly shows the aperture 107 through which either fluid or a guidewire may pass as previously described. Thus, the aperture 107 is not blocked nor is access to the lumen precluded.

With reference to FIG. 12 there is shown a cross-sectional view of the distal end of the device 120 as shown in FIG. 11 taken along line 5-5 in FIG. 11. Here, the lateral attachment portions 103a are shown at each side of the electrode 103 where electrical continuity is assured from the electrically conductive shaft to the cutting portion forming the electrode 103. Thus, the elongate member 102 is an electrically conductive tubular member where the elongate member 102 defines a lumen 109 for receiving a fluid. Any fluid within the lumen 109 may be injected, withdrawn, or may remain substantially stationary. In some embodiments, the electrically conductive tubular member is comprised of stainless steel as previously mentioned.

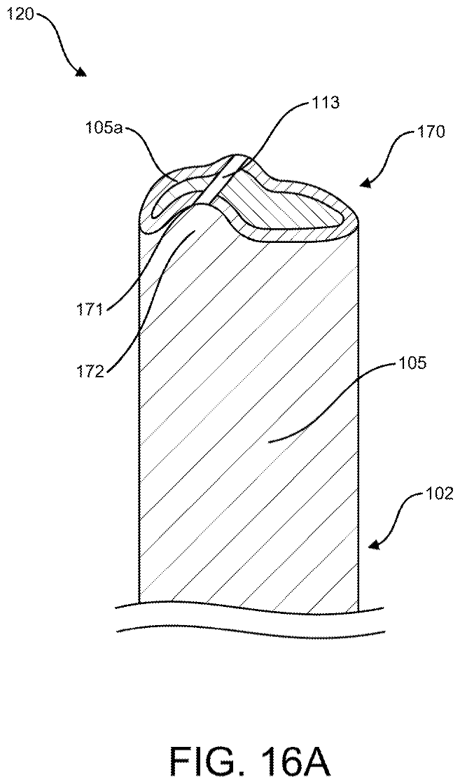

FIG. 13 and FIG. 14 relate, respectively, to the first and second embodiments of the present invention and differ in terms of the illustrated configurations of the distal face. In particular, FIG. 13 shows the arched-taper profile of the distal portion formed by the electrode 603 and distal face 604 where such arched-taper profile is embodied in the electrode 603 itself. In contrast, FIG. 14 shows the arched-taper profile of the distal portion formed by the electrode 703 and distal face 704a/704b where such arched-taper profile is embodied in the distal face 704a/704b itself. In either embodiment, an area of the distal portion therefore forms the arched-taper profile which effectively provides advantageous tapering and enables dilation of the puncture.

With particular regard to FIG. 13, there is shown a close-up perspective view of the distal end of the device 120 with its insulative layer partially removed. Here, the distal face 604 is configured as a flat ring that is perpendicular to the lengthwise axis of the overall shaft 600. As shown, the electrode 603 extends in an arched taper-profile as previously described. In this close-up however, the lateral attachment 603a of the electrode 603 is clearly visible being attached in a permanent and electrically conductive manner to the shaft 600. As before, insulative material 105 is provided upon the shaft 600 and upon the distal face 604 in order to prevent unwanted electrification of parts of the device other than the intentionally exposed portions of the electrode 603. While FIG. 13 shows a cutaway section of insulation for purposes of illustrative clarity, it should be understood that the insulative material 105 covers the entire length of the radiofrequency needle that embodies the inventive electrosurgical device, excluding the distal end of the shaft forming the electrode. As discussed, this insulative material (e.g., PTFE) is highly lubricious, allowing easy advancement/retraction of the inventive device from accessory devices and patient vasculature. The supply chain and manufacturing processes related to this insulative material are well known in the art and not further described herein. In some embodiments, the electrode 603 may include a uniform curve. In other embodiments, the electrode 603 may include a substantially flat portion that is parallel to the distal face 604. The radius of the electrode 603 is large enough to allow a guidewire or guiding member of at least 0.014 inches in diameter to be advanced through the electrosurgical device 120.

A second inventive configuration of the distal face is disclosed by way of FIG. 14. More specifically, FIG. 14 is a close-up perspective view of the distal end of a second embodiment in accordance with the present invention and with its layer partially removed. Here, the distal face is formed as two distinct and symmetrical, yet contiguous, sloped distal faces 704a and 704b. In this manner, the collective distal face comprised by sloped distal faces 704a and 704b thus forms an arched taper-profile at the distal tip of the device and which serves to gradually expand (i.e., dilate) the initial puncture hole as the shaft 700 is advanced. As shown, the electrode 703 in this second embodiment extends linearly across the distal-most surface of the collective distal face comprised by sloped distal faces 704a and 704b. As in the first embodiment, the lateral attachment 703a of the electrode 703 is clearly visible being attached in a permanent and electrically conductive manner to the shaft 700. As before, insulative material 105 is provided upon the shaft 700 and upon the sloped distal faces 704a and 704b such that unwanted electrification of parts of the device is thereby prevented other than the intentionally exposed portions of the electrode 703. While FIG. 14 shows a cutaway section of insulation for purposes of illustrative clarity, it should be again be understood that the insulative material 105 covers the entire length of the radiofrequency needle that embodies the inventive electrosurgical device, excluding the distal end of the shaft forming the electrode. As discussed, this insulative material (e.g., PTFE) is highly lubricious, allowing easy advancement/retraction of the inventive device from accessory devices and patient vasculature. Such advancement/retraction is further advantageously facilitated by the arched taper-profile configuration provided by the sloped distal faces 704a and 704b. Again, it should be understood that the supply chain and manufacturing processes related to this insulative material are well known in the art and not further described herein.

It should be understood that in either embodiment of the present invention the electrically conductive tubular member that forms the shaft is at least partially covered by electrically insulating material 105 with a distal portion of the electrically conductive radiofrequency active plate uncovered (i.e., electrically exposed) to define the given electrode 103, 603, 703. The non-cutting portion of the given distal face 104, 604, 704 includes a layer of electrical insulation, which in some embodiments may be the same as the electrically insulating material covering the shaft of the elongate member 102, which includes both the electrically insulating material 105 covering the shaft of the elongate member 102 extending up to the given distal face 104, 604, 704a/704b and the electrical insulation covering the given distal face 104, 604, 704a/704b being the same type of material applied separately.

In alternative embodiments, the layer of electrical insulation covering the given distal face 104, 604, 704a/704b may be a different type of insulation. In manufacture, the insulative coating may be applied along the circumference of the open lumen, to prevent transmission of radiofrequency energy and tissue coring. For example, the distal tip of the shaft may be dipped in paralene dielectric coating or PTFE heat-shrink may be applied, prior to application of insulation along the length of the shaft and welding of the electrode plate. In some embodiments, the non-cutting portion of the given distal face 104, 604, 704a/704b may be formed of a polymer insulation, which may be a heat shrink, a spray coating, sputtering or a material selectively coated by vapor deposition. In some alternative embodiments, non-cutting portion forming the given distal face 104, 604, 704a/704b may comprise a ceramic. In particular, non-polymeric coatings (e.g., ceramics, oxides, and diamond thin film) may function as effective insulators in thinner layers than typical polymers. In some examples of electrosurgical device 120, the electrically insulative coating comprises a layer less than about 1 micron thick. In some specific examples, the electrically insulative coating comprises a layer from about 100 nanometers to about 1 micron thick. In some other examples, the electrically insulative coating comprises a layer about 1 micron to about 50 microns thick. In some specific examples, the electrically insulative coating comprises a layer about 1 micron to about 25 microns thick, and some more specific examples, the electrically insulative coating comprises a layer about 1 micron to about 10 microns thick.

As mentioned, the given distal face 104, 604, 704 of the electrosurgical device 120 defines an aperture 107 which is in communication with lumen 111 (shown in FIG. 12). In terms of the second embodiment as shown by FIG. 14, the sloped distal faces 704a, 704b are substantially rounded or atraumatic at the distal-most portion of the device, as it is not necessary to have a sharp tip on the device for puncturing. The rounded tip reduces the risk of accidental tissue puncture and skiving of supporting dilators. In other words, the distal-most portion of the device includes sloped distal faces 704a, 704b which are substantially rounded. Furthermore, the planar surface of given distal face 104, 604, 704a/704b is substantially atraumatic.

In terms of the given electrode, it should be understood the electrode has the general configuration of a plate and is comprised of an electrically conductive material, for example, metal. In either embodiment the electrode itself has no sharp corners or edges to prevent the formation of hot spots caused by discontinuities. Overall, it should therefore be readily apparent that the present invention does not form a ring-shaped electrode capable of coring out tissue.

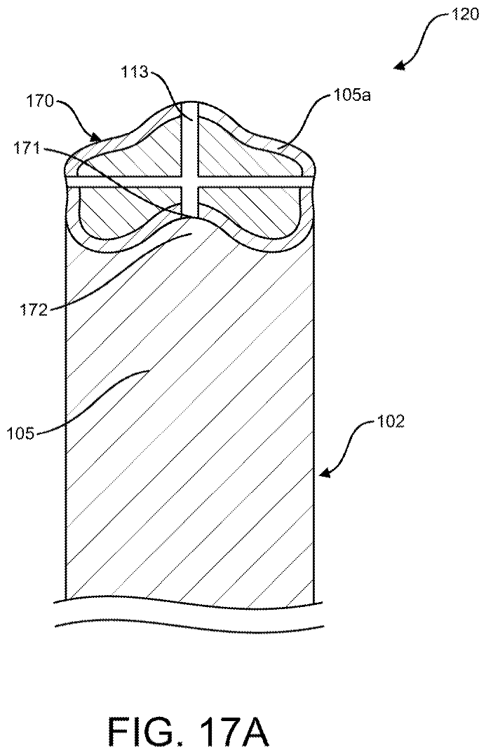

FIG. 15A illustrates an embodiment for an electrosurgical device 120 having an electrode in the form of a radiopaque radiofrequency (RF) band or ribbon 113 at the apex 171 of an arched distal tip 170. According to various embodiments, the RF band or ribbon is generally long and relatively narrow. In various exemplary embodiments, the ribbon 113 has a length equal to the diameter of the distal face 104. In some embodiments, the ribbon has a length substantially greater than the diameter of the distal face, for example two or three times greater, which allows the band to extend along and attach to an outer surface of the electrosurgical device 120. In various embodiments, the RF band or ribbon has a width of about 10% to about 50% of the diameter of the distal face 104. In various embodiments, the thickness of the RF band or ribbon is less than the width. In various embodiments, the RF band or ribbon has a thickness sufficient to provide structural support during the tissue crossing procedure, while maintaining substantial flexibility. In some embodiments, the RF band or ribbon has a thickness of from about 0.04 millimeters to about 0.75 millimeters.

The arched distal tip 170 includes a plurality of arches 172, each of the plurality of arches 172 including an apex 171. An RF ribbon 113 is joined to the distal face 104 of each apex 171, for example by welding. The RF ribbon 113 is electrically connected to each apex 171 in order to receive energy from an energy source connected to a proximal end of the electrosurgical device 120. The RF ribbon 113 acts as an electrode, which administers energy to puncture tissue when placed against a target tissue.

The elongate member 102 is composed of 304 Stainless Steel. 304 Stainless Steel is biocompatible, conductive, and possess suitable material properties (e.g. stiffness). An insulative coating 105 is applied along the circumference of the elongate member 102, to prevent transmission of RF energy and tissue coring. The arched distal tip 170 may be dipped in Paralene dielectric coating or PTFE heat-shrink may be applied, prior to application of insulation 105 along the length of the elongate member 102 and connection of the RF ribbon 113 to each apex 171. In some embodiments, the RF ribbon 113 may be welded or bonded to each apex 171 using an electrically conductive adhesive. The distal face 104 of the arched distal tip 170 is electrically insulated, and forms the non-cutting portion of the distal end.

The insulation 105 covers the entire length of the electrosurgical device 120, excluding the arch distal tip 170. The insulation 105 material can be highly lubricious, allowing easy advancement and retraction of the electrosurgical device 120 within accessory devices and patient vasculature. In FIG. 15A and 15B, insulated portions of the electrosurgical device 120 are illustrated by cross-hatching while uninsulated active regions do not include cross-hatching.

The electrosurgical device 120 is intended for use in minimally invasive cardiac procedures, allowing surgeons to gain transseptal access by puncturing the fossa ovalis in the heart. The arched distal tip 170 allows for gradual expansion of an initial puncture hole within tissue. This is essential for preventing tissue coring and mitigating the risk of introducing free-floating particles into the patient anatomy. The arched geometry at the distal tip is also critical for allowing a guide-wire of approximately 0.014 inches in diameter, to advance through a lumen of the electrosurgical device 120 and anchor the puncture site during initial crossing of the septum. The RF ribbon 113 is used to create the initial puncture in the tissue. The electrosurgical device 120 may be applicable to similar areas of use, provided that the requirements and constraints of the procedure are similar to that of a minimally invasive transseptal access cardiac surgery.

The arched distal tip 170 can be manufactured using CNC machining techniques, metal injection molding (MIM), cold forming or other techniques suitable to create the arches 172.

In some aspects, the RF ribbon 113 may be composed entirely of platinum or 304 stainless steel plated with platinum. The use of platinum, or an equivalently suitable material, ensures the RF ribbon 113 is radiopaque and can be visualized under fluoroscopy and echocardiography. The RF ribbon 113 can be fixed to the apex 171 of the arched distal tip 170 using micro laser welding or similar techniques. The RF ribbon 113 includes a width that is a minimum of 0.008 inches. The RF ribbon 113 may have a generally flat configuration. The RF ribbon 113 may include a cross-section that is substantially rectangular, or may include a cross-section having curvature, such as a half-dome. The cross-sectional shape of the RF ribbon 113 can also include an oval, circle, or polygon.

FIG. 15B is a top view of the electrosurgical device 120 of FIG. 15A. As shown, the elongate member 120 includes a circular cross-section. This distal face 104 of the arched distal tip 170 forms the non-cutting portion 105a, and appears as a circle when looked at from above. The RF ribbon 113 transverses the arched distal tip 170 along a midline of the circular cross-section. The RF ribbon 113 divides the aperture 107 into two equal portions. In some aspects, the arches of the distal tip 170 may be positioned off center, such that the RF ribbon 113 divides the aperture 170 into two non-equal portions. In some aspects, the arched distal tip 170 may include more than two arches, and the RF ribbon 113 may divide the aperture 170 into more than two equal portions.

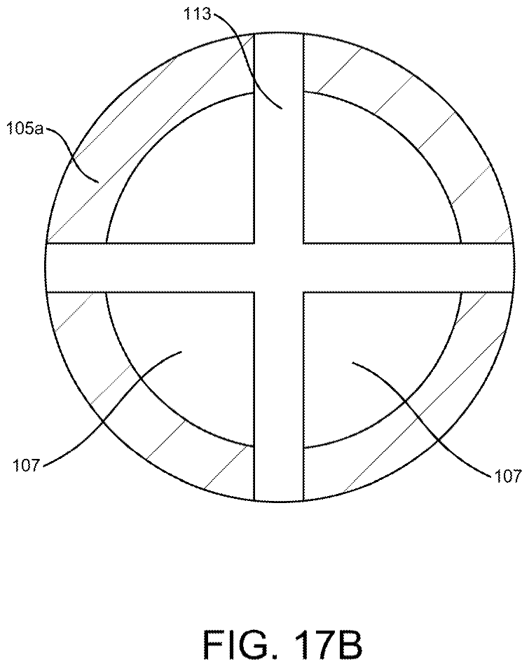

FIG. 16A illustrates a perspective view of an embodiment of an electrosurgical device 120 having an arched distal tip 170, wherein the arches 170 are positioned at locations offset from a central axis of the arched distal tip 170. In this configuration, the RF ribbon 113, when joined to each apex 171, is off center and divides the aperture into two non-equal portions. This can provide room such that a larger guide-wire or other guiding member can be advanced through the arched distal tip 170.

FIG. 16B is a top view of the electrosurgical device 120 of FIG. 16A. The RF ribbon 113 divides the aperture into a first larger aperture 107a and a second smaller aperture 107b. In FIGS. 8C and 8D, insulated portions of the electrosurgical device 120 are illustrated by cross-hatching while uninsulated active regions do not include cross-hatching.

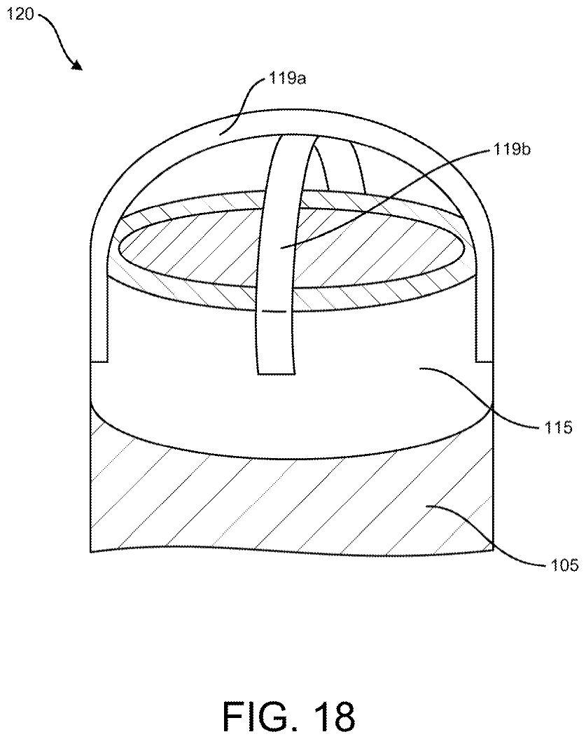

FIG. 17A illustrates a perspective view of an embodiment of an electrosurgical device 120 having an arched distal tip 170 including multiple sets of arches 170. In the embodiment of FIG. 17A, the arched distal tip 170 includes four arches 170.

The arches 170 are located equidistant apart around the circumference of the arched distal tip 170. In this configuration, the RF ribbon 113 divides the aperture into four equal portions when joined to the apex 171 of each arch 170. FIG. 17B is a top view of the electrosurgical device 120 of FIG. 17A. The RF ribbon 113 can be formed from a single piece of material in the shape of a cross or an “x” or can be formed from two separate ribbons joined together. In FIGS. 17A and 17B, insulated portions of the electrosurgical device 120 are illustrated by cross-hatching while uninsulated active regions do not include cross-hatching.

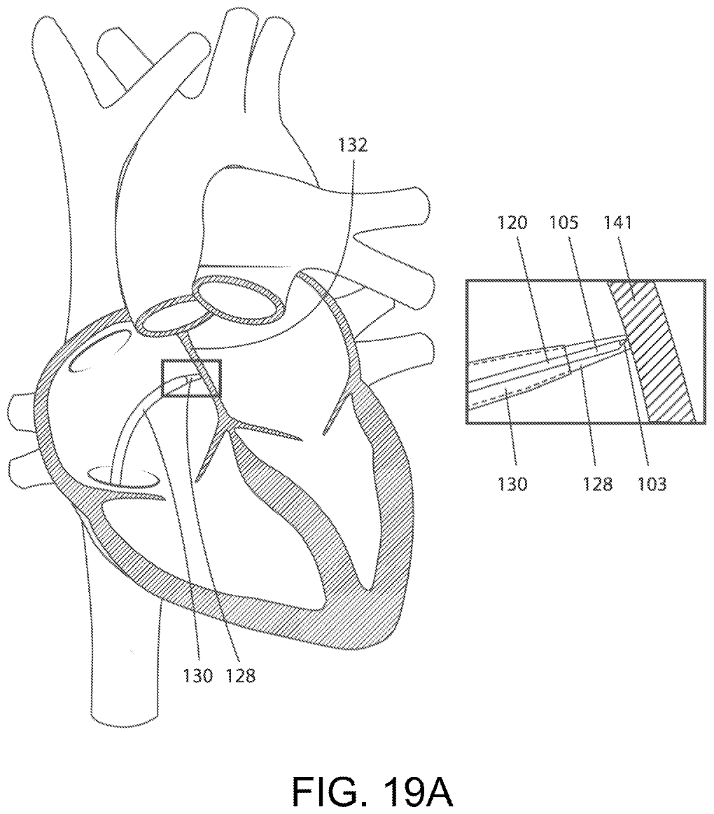

FIG. 18 illustrates an embodiment for an electrosurgical device 120 having with multiple arched RF ribbons 119a, 119b at the distal end of the electrosurgical device 120 as opposed to an arched distal tip. The arched RF ribbons 119a, 119b can be fixed to an exposed proximal portion 115 using micro laser welding techniques. The arched RF ribbons 119a, 119b can be positioned substantially orthogonal to each other. In one embodiment, each of the RF ribbons 119a, 119b are formed as separate pieces of a flat material. In another embodiment, the RF ribbons 119a, 119b are formed of a single piece of material in the form of an “x” or cross. In FIG. 18, insulated portions of the electrosurgical device 120 are illustrated by cross-hatching while uninsulated active regions do not include cross-hatching.

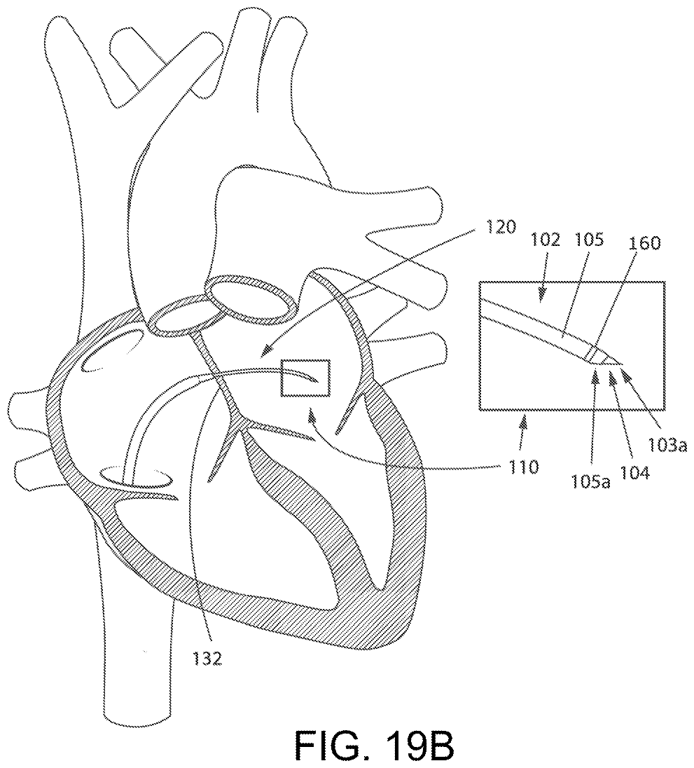

FIGS. 19A and 19B illustrate an embodiment of a method of puncturing tissue. The method comprises the steps of (a) delivering energy through electrically exposed conductive portion 103a of electrosurgical device 120 to tissue 141 at a target site for creating a puncture substantially corresponding to an elongate cutting portion of the distal face of the electrosurgical device; and (b) dilating or widening the puncture primarily by advancing a flat-tipped or angled distal surface of the electrosurgical device, without coring the tissue. In some embodiments the step of delivering energy comprises creating a flap in the tissue and the step of dilating or widening is completed without further delivery of energy. In some embodiments, the target site is a tissue within a heart, and in some particular embodiments the tissue is an atrial septum 132. Typically, the method uses a sheath. The term dilate is used herein to mean “to make wider, larger, or more open”.

An alternative embodiment of a method of puncturing tissue comprises the steps of (a) delivering energy through a cutting portion a distal face of an electrosurgical device to tissue at a target site to create an elongate puncture through the tissue, while preventing delivery of energy from a non-cutting portion of the distal face; and (b) advancing the electrosurgical device through the tissue by pushing aside a flap of tissue defined by the puncture. The step of delivering energy comprises creating a slit or slits in the tissue.

Dilating the puncture typically includes displacing the tissue. In some embodiments dilation includes wedging apart and thereby outwardly compressing surrounding portions of the tissue.

Some embodiments of the method include using a medical imaging modality to guide the electrosurgical device 120 to the target site. Some embodiments comprise measuring pressure for positioning electrosurgical device 120 at the target site. In some embodiments, the method includes using a radiopaque marker 160 for positioning electrosurgical device 120. Some embodiments include advancing the electrosurgical device to the target site over a guide-wire.

In some embodiments, the method includes advancing electrosurgical device 120 to the target site through a dilator 128; positioning electrosurgical device 120 such that cutting portion 103a is aligned with or protruding slightly from a distal end of the dilator 128; and delivering fluid through an aperture 107 (e.g. FIG. 2A) at a distal end of electrosurgical device 120 to stain the tissue. The fluid is typically delivered longitudinally forward through the electrosurgical device. Some embodiments further comprise a step of withdrawing a fluid via an open distal face of the electrosurgical device.

In some embodiments, the distal surface of the electrically exposed conductive portion 103a is generally C-shaped and step (b) includes creating a generally C-shaped puncture. In some other embodiments, the distal surface of the electrically exposed conductive portion is generally crescent-shaped and step (b) includes creating a generally crescent-shaped puncture. In yet other embodiments, the distal surface of the electrically exposed conductive portion is generally arcuate-shaped and step (b) includes creating a generally arcuate-shaped puncture.

In some embodiments of the broad aspect, the aperture 107 and the lumen 111 together comprise a pressure transmitting lumen, and the method further comprises measuring a fluid pressure of the pressure transmitting lumen using a pressure sensing mechanism.

In an RF perforation or puncturing procedure, unlike RF ablation, energy is applied to rapidly increase tissue temperature to the extent that the intracellular fluid becomes converted to steam, inducing cell lysis as a result of elevated pressure within the cell. Upon the occurrence of cell lysis and rupture, a void is created, allowing the tip of the catheter to penetrate the tissue. In order to achieve this effect, RF perforation devices must apply a high voltage to the tissue region over a short period of time. Also, the tip of the device being used should be relatively small, in order to increase the impedance of the device. This is in contrast to RF ablation, whereby a larger-tipped device is utilized to deliver a low impedance and high power signal to the region involved. Furthermore, as opposed to RF perforation, which creates a void in the tissue through which the device may be advanced, the objective of RF ablation is to create a large, non-penetrating lesion in the tissue, in order to disrupt electrical conduction. Thus, for the purposes of the present invention, perforation is defined as the creation of a void within a material.

Embodiments of the present invention are operable to create such punctures or voids without substantially removing a plug or core of material from the tissue at the target site, since the puncture resulting from devices as described hereinabove are typically slit-like, C-shaped, or similar configurations substantially corresponding to the shape(s) of the cutting portion of the distal face of the electrosurgical device.

Electrosurgical device 120 may be used in conjunction with a source of radiofrequency energy suitable for perforating material within a patient's body. The source of energy may be a radiofrequency (RF) electrical generator, operable in the range of about 100 kHz to about 1000 kHz, and designed to generate a high voltage over a short period of time. More specifically, in some embodiments, the voltage generated by the generator increases from about 0 V (peak-to-peak) to greater than about 75 V (peak-to-peak) in less than about 0.6 seconds. The maximum voltage generated by generator may be between about 180V peak-to-peak and about 3000V peak-to-peak. The waveform generated may vary, and may include, for example, a sine-wave, a rectangular-wave, or a pulsed rectangular wave, amongst others. During delivery of radiofrequency energy, the impedance load may increase due to tissue lesioning near the target-site, or the formation of a vapor layer following cell rupture, for example. The generator may be operable to continue to increase the voltage, even as the impedance load increases. For example, energy may be delivered to a tissue within a body at a voltage that rapidly increases from about 0 V (RMS) to about 220 V (RMS) for a period of between about 0.5 seconds and about 5 seconds.

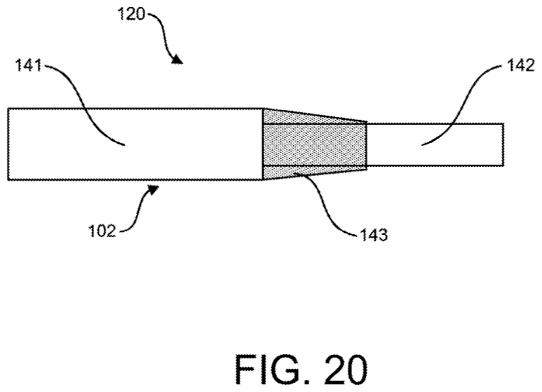

FIG. 20 illustrates a manufacturing process for joining two separate hypotubes 141, 142 to form an elongate member 102 of an electrosurgical device 120 in accordance with an embodiment of the disclosure.

In some aspects, the elongate member 102 is composed of two separate hypotubes, a proximal hypotube 141 and a smaller distal hypotube 142. In order to reduce the risk of bent or broken tip failure, a tapered socket 143 can be included at the joint where the proximal hypotube 141 and the distal hypotube 142 meet. The tapered socket 143 may be glued or shrink-fit to the joint, prior to application of PTFE heat-shrink insulation as discussed previously. The taper length of the tapered socket 143 must be enough to minimize the risk of fracture at the proximal-distal joint and along the socket boundaries. The tapered socket 143 may be composed of 304 stainless steel (or equivalent).

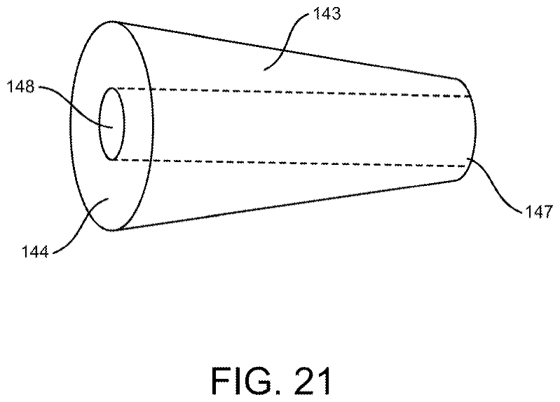

FIG. 21. Illustrates a tapered socket 143 that can strengthen the joint between the two separate hypotubes in accordance with an embodiment of the disclosure. The tapered socket 143 includes a proximal end 144 and a distal end 147. A lumen 148 extends from proximal end 144 to the distal end 147 and is configured to receive a hypotube.

In some aspects, the elongate member 102 is composed of single tapered hypotube. The tapered geometry is necessary in order to ensure electrosurgical device 120 is compatible with accessory devices such as a catheter, sheath, or dilator.

Without being limited to a particular theory of operation, it is believed that under particular circumstances, for example as mentioned hereinabove, dielectric breakdown and arcing may occur upon the delivery of radiofrequency energy, whereby polar molecules may be pulled apart. The combination of these factors may result in the creation of an insulative vapor layer around the electrode, therein resulting in an increase in impedance, for example the impedance may increase to greater than 4000 Ω. In some embodiments, despite this high impedance, the voltage continues to increase. Further increasing the voltage increases the intensity of fulguration, which may be desirable as it allows for an increased perforation rate and puncture creation. An example of an appropriate generator for this application is the BMC RF Perforation Generator (model number RFP-100A, Baylis Medical Company, Montreal, Canada). This generator delivers continuous RF energy at about 460 kHz.

A grounding pad or dispersive electrode may be electrically coupled to the generator for contacting or attaching to the body of the patient to provide a return path for the RF energy when the generator is operated in a monopolar mode.

Additional details regarding the device and method may be found in U.S. application Ser. No. 13/468,939, filed May 10, 2012, U.S. application Ser. No. 11/905,447, filed Oct. 1, 2007 (now issued as U.S. Pat. No. 8,192,425), U.S. application Ser. No. 13/113,326, filed May 23, 2007, U.S. application Ser. No. 11/265,304, filed Nov. 3, 2005 (now U.S. Pat. No. 7,947,040), U.S. application Ser. No. 10/666,301, filed Sep. 19, 2003 (now issued as U.S. Pat. No. 7,048,733), U.S. application Ser. No. 10/760,479, filed Jan. 21, 2004 (now issued as U.S. Pat. No. 7,270,662), U.S. application Ser. No. 10/666,288, filed Sep. 19, 2003, U.S. application Ser. No. 10/347,366, filed Jan. 21, 2003 (now issued as U.S. Pat. No. 7,112,197), U.S. provisional application Ser. No. 60/522,753, filed Nov. 3, 2004, and provisional applications Ser. No. 60/884,285, filed Jan. 10, 2007, 60/827,452, filed Sep. 29, 2006, Ser. No. 61/653967, filed May 31, 2012, and Ser. No. 61/681,512, filed Aug. 9, 2012. The contents of all above-named applications and patents are incorporated herein by reference in their entirety.

Thus, as described hereinabove, the problem of puncturing tissue without coring, while providing forward fluid delivery, is solved by an electrosurgical device comprising a distal face defining at least one aperture, and the distal face including at least one cutting portion and at least one non-cutting portion cooperating to produce an elongated cut in a tissue when electrical energy is delivered to the distal face, while avoiding coring of the tissue.

Various modifications and additions can be made to the exemplary embodiments discussed without departing from the scope of the present invention. For example, while the embodiments described above refer to particular features, the scope of this invention also includes embodiments having different combinations of features and embodiments that do not include all of the described features. Accordingly, the scope of the present invention is intended to embrace all such alternatives, modifications, and variations as fall within the scope of the claims, together with all equivalents thereof.

We claim:

1. An electrosurgical device for puncturing a tissue, the device comprising:
an elongate body having a proximal portion and a distal portion, and a lumen extending from the proximal portion to the distal portion;
a connector for connecting with an energy source;
the distal portion including an arched distal end, wherein the arched distal end includes a first apex and a second apex; and
an RF ribbon configured to deliver energy for puncturing the tissue, wherein the RF ribbon is in electrical communication with the first apex and the second apex.

2. The device of claim 1, wherein RF ribbon is radiopaque.

3. The device of claim 1, wherein arched distal end includes a third apex and a fourth apex.

4. The device of claim 1, wherein the distal portion includes an aperture, and the RF ribbon separates the aperture into equal portions.

5. The device of claim 1, wherein the distal portion includes an aperture, and the RF ribbon separates the aperture into unequal portions.

6. The device of claim 1, wherein the elongate body is formed of a first hypotube and a second hypotube.

7. The device of claim 6, further comprising a tapered socket positioned at a joint reinforcing the first hypotube and the second hypotube.

8. The device of claim 1, wherein the elongate body is formed of a hypotube that decreases in diameter towards the arched distal end.

9. The device of claim 1, wherein the RF ribbon is joined to a distal face of the first apex and the second apex.

10. The device of claim 1, wherein the RF ribbon is joined to a side of the arched distal end.

11. The device of claim 1, wherein the RF ribbon includes an arc.

12. The device of claim 1, wherein the arched distal end includes insulation.

13. The device of claim 12, wherein the insulation is a coating or heat shrink material, and wherein the coating or heat shrink material includes PTFE, Paralene, oxides, nitrides, or ceramics.

14. The device of claim 1, wherein the arched distal end includes a distal insulated portion and an exposed proximal portion, and the RF ribbon is joined to the exposed proximal portion.

15. The device of claim 1, wherein the lumen includes an inner surface, and insulation is positioned along the inner surface.

16. An electrosurgical device for puncturing a tissue comprising:
an elongate member defining a lumen for fluid;
a distal portion which includes an electrode and a distal face, the electrode being located centrally on the distal face;
the distal face defining at least one aperture and including two symmetrical openings located adjacent to the electrode;
the distal portion including at least one non-cutting portion and at least one cutting portion configured to deliver energy for puncturing the tissue wherein a distal surface of the electrode forms the at least one cutting portion; and
an area of the distal portion defining an arched-taper profile located about the at least one aperture.

17. The electrosurgical device of claim 16, wherein the radiofrequency active plate is permanently affixed to electrically conductive tube the electrically conductive portion of the elongate member.

18. The electrosurgical device of claim 17, wherein the two symmetrical openings are dimensioned to allow passage of a guidewire.

19. An electrosurgical device for puncturing a tissue comprising:
an elongate member defining a lumen for fluid;
a distal portion which includes an electrode and a distal face, the electrode formed by a radiofrequency active plate and permanently affixed centrally upon the distal face;
the distal face defining at least one aperture and including two symmetrical openings located adjacent to the electrode, the openings capable of allowing unimpeded passage therethrough;
the distal portion including at least one non-cutting portion and at least one cutting portion formed by the radiofrequency active plate for puncturing the tissue; and
an area of the distal portion defining an arched-taper profile located about the at least one aperture.

20. The electrosurgical device of claim 19, wherein the distal face forms an arched-taper profile, and the radiofrequency active plate is located at a distal-most location of the elongate member.
